(19) United States
(12) Patent Application Publication
(10) Pub. No.: US 2011/0166098 A1
(43) Pub. Date: Jul. 7, 2011

(54) MIXTURE OF HYALURONIC ACID FOR TREATING AND PREVENTING INFLAMMATORY BOWEL DISEASE

(76) Inventor: Tsung-Chung WU, Taipei County (TW)

(21) Appl. No.: 12/651,712

(22) Filed: Jan. 4, 2010

(51) Int. Cl. A61K 31/728; A61P 1/04; A61P 1/06

(52) U.S. Cl. 514/54

(57) ABSTRACT

The present invention is related to a hyaluronic acid formulation including a mixture of hyluronic acids having different weight-average molecular weight and different rheological, tissue scaffold and degradation properties in aqueous solution. The resulting formulation demonstrated an optimal balance between adhesion, tissue scaffold and treating time on the treatment and prevention of IBD (inflammatory bowel disease) such as ulcerative colitis and Cohn's disease. Thus, the formulation of the present invention exhibits a quick and lasting effect on the treatment and prevention of duodenal or peptic ulcer and bleeding which is very good thing indeed.

### BACKGROUND OF THE INVENTION

1. Field of the Invention

The present invention provides a mixture of the hyaluronic acid for treating and preventing the inflammatory bowel disease (IBD). More particularly, the present invention relates to a mixture comprising at least two or more than two different average hyaluronic acid molecular weights (Mw) and hyaluronic acid with different rheology to gain a hyaluronic acid with the proper adhesion property, functions of tissue scaffold and treatment time, in order to treat and to prevent IBD (inflammatory bowel disease) includes ulcerative colitis and Cohn's disease, thus to achieve the prompt treatment and to prolong the effect.

2. Description of Related Art

Hyaluronic acid also known as hyaluronan, hyaluronate and sodium hyaluronate, and generally referred to as HA, which is a natural glycosaminoglycan including the alternative N-acetyl-D-glucosamine and D-glucuronic acid moiety.

The macromolecule of hyaluronic acid in sodium salt form generally is the known composition existing for over fifty years. Referring to Meyer and al (J. Biol Chem. 107,629 (1934)), the hyaluronic acid intrinsically contains the high-viscosity glycosamine alternative with β 1-3 glycuronic acid and β 1-4 glycosamine, and the Mw of the high-viscosity glycosamine is between 50,000 Dalton (Da) and few million Dalton.

We can find the hyaluronic acid in the soft connective tissue in the body of mammals, and the skin, the vitreous humor of the eye, the synovial fluid, the umbilical cord and cartilage tissue contains higher volume of the hyaluronic acid.

The hyaluronic acid is the fluid with the elasticity, filling between the cells and the collagenous fibers and covering onto some epidermal tissues, majorly for the protection and the lubricant to cells for providing a platform for transporting the regulatory T cell to stabilize and to protect collagen network from the mechanical damage. The hyaluronic acid can be the lubricant in the tendon and the tendon sheath and on the surface of the synovial membrane due to the lubricant feature and the high shock absorber, and it is helpful for the tissue rheological mechanics, motion and the cell proliferation (referring to Delpech, B., Girard, N., Bertrand, P., Courel, M.-N., Chauzy, C., Delpech, A., 1997. Hyaluronan: fundamental principles and applications in cancer. J. Intern. Med. 242,41-48, Rooney, P., Kumar, S., Ponting, J., Wang, M., 1995. The role of hyaluronan in tumour neovascularization. Int. J. Cancer 60, 632-36, Entwistle J, Hall C L, Turley EA Receptors: regulators of signaling to the cytoskeleton. J Cell Biochem 1996; 61: 569-77), and participating the receptor interaction on the surface of some cells; especially the major receptor of CD44. The regulatory effect of CD44 is widely accepted as a mark of the activated lymphocyte (referring to Teder P, Vandivier R W, Jiang D, Liang J, Cohn L, Pure E, Henson P M, Noble P W. Resolution of lung inflammation by CD44. Science 2002; 296: 155-158, Sheehan K M, DeLott L B, Day S M, DeHeer D H, Hyalgan has a dose-dependent differential effect on macrophage proliferation and cell death. J Orthop Res 2003; 21: 744-51).

The hyaluronic acid has the ability of creating and filling due to the organization and modification of the extracellular matrix, and is widely applied in filling the soft tissue for restraining the skin aging caused by age and light, as well as to adjust the obstacle of lipid metabolism on face, to prevent the increasing of secondary scar or scar formation on the skin.

Furthermore, the hyaluronic acid can be applied as the adjuvant agent for the eye operation or to reduce the pain while moving the knee and joint of the osteoarthritis patients.

Recently, the hyaluronic acid is applied in clinical treatment in the sodium salt form majorly in eye, skin, surgeon, artery treatment and in cosmetic fields. The hyaluronic acid with alkali metal ion, alkaline earth metal ion (for example the magnesium ion), aluminum ion, ammonium ion, and salt form of the replacement of the ammonium ion can be the carrier for assisting drug absorption (referring to Belgium Patent 904,547). The silver salt is used as the mycocide and the gold salt is used for treating the rheumatoid arthritis among the heavy metal salt of the hyaluronic acid (referring to WO 87/05517).

The effect of treating the hipsore and the decubitus by the composition (complex) of the hyaluronic acid and the metal ion in the fourth group of periodic table, for example the zinc hyaluronate and the cobalt salt have been proven in the Hungary Patent 203,372 to the world.

Bioniche, the Canadian company, disclosed a method and related structure for using the hyaluronic acid with an effective concentration to treat cystitis in U.S. Pat. No. 5,888,986, wherein the Mw of the hyaluronic acid is more than 200,000 Da. There is only the hyaluronic acid with the certain Mw been applied in the embodiment thereof, for example, to use the hyaluronic acid with the 650 kDa or 1,900 kDa Mw to treat the cystitis; however, the single molecular weight of the hyaluronic acid can not be used for both prompt treatment and sustained effect.

The US patent application 2005/0080037 (A1) belonging to Robert Peter Petrella, a Canadian, disclosed the use of hyaluronic acid for treating acute and over sprain and the reaction thereof, wherein the Mw of the hyaluronic acid is only between 90 thousand Da to 120 Da, and a single molecular weight of the hyaluronic acid cannot perform both prompt healing and prolonged action.

Seikagaku Kogyo, a Japanese company, has filed a U.S. Pat. No. 7,354,910 on Apr. 4, 2008 entitled “Use of Agent for treating inflammatory bowel disease” to disclose that the hyaluronic acid and hyaluronate with Mw between 600 kDa and 1,200 kDa can be applied to treat IBD. However, the degradation is too fast to retain the treating effect after injecting into the patient, therefore, it's very inconvenient for patients clinically.

### SUMMARY OF THE INVENTION

An object of the present invention is to use the biological activity of at least two or more than two average molecular weight of hyaluronic acids in the pharmaceutically acceptable salt with different Mw to treat IBD. Because the low average molecular weight hyaluronic acid (LMWHA) and the high average molecular weight hyaluronic acid (HMWHA) have different adhesive and degradation rate, the hyaluronic acid with average Mw lower than 1.5 million Da is categorized as LMWHA, and the higher than 1.5 million Da is categorized as HMWHA. Thus, mixture of LMWHA and HMWHA can form a desired formulation, wherein the LMWHA can rapidly cover the inflammatory surface to treat IBD (for example, ulcerative colitis, acute enteritis, chronic enteritis or Cohn's disease), and the HMWHA can prolong the degradation in order to achieve a longer effective period. Thus, a faster treatment and a sustained release effect may be achieved.

Another object of the present invention is to provide a hyaluronic acid mixture comprising both LMWHA and HMWHA together with a steroid, immunosuppressive agent or other anti-inflammatory drug in order to potentiate the effect.

Another object of the present invention is to provide a hyaluronic acid mixture comprising both LMWHA and HMWHA forming the major active ingredient with the proper excipient to formulate as tablet, suppository, endoscopic injection or rectal perfusion (for example enema).

Another object of the present invention is to provide a preferred concentration of a hyaluronic acid mixture comprising both LMWHA and HMWHA or the pharmaceutically acceptable salt thereof in a range from 0.5 mg/ml to 5 mg/ml, and a more preferable concentration in the solution form is in a range from 0.05% to 0.5% (w/v).

### DETAIL DESCRIPTION OF THE INVENTION

The hyaluronic acid mixture of the present invention used to treat and to prevent the IBD comprises using at least two or more than two average molecular weight of hyaluronic acids including mixture of low average molecular weight hyaluronic acid (LMWHA) and high average molecular weight hyaluronic acid (HMWHA). Different Mw has different rheology, functions of tissue scaffold and degradation in the solution, and therefore, the hyaluronic acid mixture can balance the drug effect and the degradation rate in order to treat and to prevent IBD (for example, ulcerative colitis, acute colitis, chronic colitis or Cohn's disease), as well as to achieve a proper treatment effect and a prolonged treatment effect.

The average Mw lower than 1.5 million Da is categorized as LMWHA, and preferable within a range between 0.5 million to 1.5 million Da. The average Mw higher than 1.5 million Da is categorized as HMWHA, and preferable within a range between 1.5 million to 3.5 million Da. The formulation containing a mixture of LMWHA and HMWHA, wherein the LMWHA promptly covers the inflammatory portion to treat and to prevent IBD, and the HMWHA extends the treatment effect. Thus, achieve prompt treatment and sustained release effect.

The general chemical structure of the hyaluronan may be illustrated as follows.

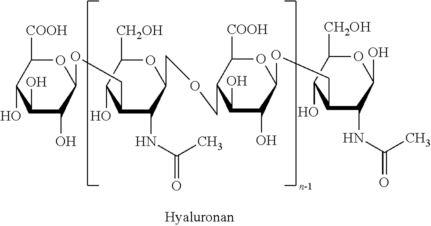

Another preferred embodiment of the present invention includes a 1: 1 mixture of LMWHA and HMWHA in a salt form of the hyaluronic acid, and the ratio may be adjusted depending on the clinical purpose between 20:80 and 80:20. The hyaluronic acid mixture with a higher ratio of LMWHA can be more helpful in speeding up the treatment; on the contrary, a higher ratio of HMWHA can provide a better degradation rate to prolong the treatment effect.

Another preferred embodiment of the present invention includes a hyaluronic acid mixture including LMWHA and HMWHA together with a steroid, immunosuppressive agent or other anti-inflammatory drug to potentiate the effect.

Another preferred embodiment of the present invention includes a hyaluronic acid mixture including both LMWHA and HMWHA constituting the major active ingredient with the proper excipient to formulate an oral solid dosage form (for example enteric coating), suppository, endoscopic injection or perfusion (for example enema).

For oral formulation (for example enteric coated tablet), the enteric coating provides more resistance dissolution and digestion in the stomach, and after reaching intestine and colon, the enteric coating will be dissolved and the hyaluronic acid will be released to form a protection membrane at the inflammatory colon (the region uprising ascending or Transverse colon) in order to accelerate healing of the inflammatory region and also to prolong the treatment effect by long degradation rate.

For suppository formulation, the suppository containing above hyaluronic acid may be inserted into the anus and the hyaluronic acid will dissolve in the rectum and spread to other region of colon (for example the descending region) to form a protection membrane at the inflammatory colon in order to accelerate healing of the inflammatory region and also achieve sustained release effect.

For perfusion formulation (for example enema), the above hyaluronic acid mixture is the major active ingredient mixed with the excipient (for example phosphate buffered saline (PBS solution or suspension formulation)) directly used or in a soft tube to inject the above hyaluronic acid mixture into the colon. The hyaluronic acid mixture will be charged into the colon and spread to other region of colon (for example the descending region) to form a protection membrane at the inflammatory colon in order to accelerate the healing of the inflamed region and also achieve sustained release effect.

The preferred concentration of the hyaluronic acid mixture including LMWHA and HMWHA or pharmaceutically acceptable salt thereof ranges from 0.5 mg/ml to 5 mg/ml, but the preferred concentration in the solution form is within a range from 0.05% to 0.5% (w/v). Furthermore, as for oral administration, the preferred dose of the present invention for prevention or improvement includes at least 10 to 100 mg for each administration; the most preferred dose is about 10 to 40 mg. The pharmaceutically acceptable salt of the hyaluronic acid mixture is the sodium hyaluronan and the zinc hyaluronan.

According to the above description, at least two or more than two average molecular weight of pharmaceutically acceptable salt of the hyaluronic acid may be with different Mw may be mixed to rapidly cover the inflammatory surface and shorten the treatment period and prolong the degradation in order to prolong the coverage period. Thus, achieve a faster treatment and sustained release effect.

While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations in which fall within the spirit and scope of the included claims. All matters set forth herein or shown in the accompanying drawings are to be interpreted in an illustrative and non-limiting sense.

What the invention claimed is:

1. A mixture of the hyaluronic acid for treating and preventing inflammatory bowel disease (IBD), comprising at least two or more than two types of average molecular weight of hyaluronic acid including a low average molecule weight hyaluronic acid (LMWHA) and a high average molecule weight hyaluronic acid (HMWHA); wherein an average molecular weight (Mw) of said LMWHA is lower than 1.5 million Da, and a Mw of said HMWHA is higher than 1.5 million Da; and a mixing ratio of said LMWHA and said HMWHA is within a range of 20:80 to 80:20.

2. A mixture of the hyaluronic acid for treating and preventing inflammatory bowel disease (IBD) according to claim 1, wherein said mixing ratio of said LMWHA and HMWHA is 1:1.

3. A mixture of the hyaluronic acid for treating and preventing inflammatory bowel disease (IBD) according to claim 1, wherein said hyaluronic acid mixture is in a concentration of 0.5 mg/ml to 5 mg/ml.

4. A mixture of the hyaluronic acid for treating and preventing inflammatory bowel disease (IBD) according to claim 1, wherein said hyaluronic acid mixture includes an excipient to formulate an oral solid dosage form, suppositories, or a suspension for oral, rectal or perfusion administration.

5. A mixture of the hyaluronic acid for treating and preventing inflammatory bowel disease (IBD) according to claim 1, wherein said hyaluronic acid mixture can be formulated together with a steroid, immunosuppressive agent or other anti-inflammatory as an adjuvant.

6. A mixture of the hyaluronic acid for treating and preventing inflammatory bowel disease (IBD) according to claim 1, wherein said hyaluronic acid can treat and prevent ulcerative colitis, acute enteritis, chronic enteritis or Cohn's disease.
